(19) United States
(12) Patent Application Publication
(10) Pub. No.: US 2022/0262275 A1
(43) Pub. Date: Aug. 18, 2022

(54) CPR TRAINING MANIKIN

(71) Applicant: AA-REA GmbH, Koeln (DE)

(72) Inventor: Bernd W. Boettiger, Köln (DE)

(21) Appl. No.: 17/621,968

(22) PCT Filed: Jun. 26, 2020
(86) PCT No.: PCT/DE2020/100557

§ 371 Date: Dec. 22, 2021

(30) Foreign Application Priority Data
Jun. 27, 2019 (DE) 10 2019 117 441.4

(51) Int. Cl. G09B 23/28; A61H 31/00

(52) U.S. Cl. CPC G09B 23/288; A61M 16/0051; A61H 31/005

(57) ABSTRACT

The invention relates to a training manikin for simulating cardiopulmonary resuscitation, comprising a foam body which supports the schematic structure of a human torso with or without a head and consists of a single foam, wherein a signal-generating unit is arranged inside the foam body and is situated in the foam body in a region underneath the sternum of the torso with or without a head and emits a signal within a time interval of 100 ms to 1000 ms when the sternum region of the torso with or without a head is compressed to a compression depth of 3 to 8 cm during a compression period. The invention also relates to the use of such a training manikin as a training device for cardiopulmonary resuscitation exercises.

The invention relates to a training manikin for simulating cardiopulmonary resuscitation, comprising a foam body which supports a schematic structure of a human torso with or without a head and consists of a singular foam, a signal-generating unit being arranged inside the foam body, which signal-generating unit is located in the foam body in a region underneath the sternum of the torso with or without a head and emits a signal when the sternum region of the torso with or without a head is compressed to a compression depth of 3 cm to 8 cm with a compression duration in a time interval of 100 ms to 1000 ms, as well as the use of such a training manikin as a training device for cardiopulmonary resuscitation exercises.

More than 100,000 additional lives could be saved each year in Europe if more medical laypersons were trained in cardiopulmonary resuscitation (CPR) techniques. The available training material, in particular the manikins provided for training, is essential for training people. However, CPR training manikins have been comparatively expensive until now. If the CPR training is intended to reach the breadth of the population, in particular schoolchildren, the costs for schools and school authorities (generally speaking, local authorities) have previously been too high for the sustainable introduction of resuscitation training.

The US 2007/0292828 A1, for example, is known as prior art, which describes a training manikin for cardiopulmonary resuscitation (CPR) that has a structure having a plurality of foam layers which are attached to one another and which are cut in the shape of a human torso with or without a head. A sound-generating unit is also disclosed which is arranged in a portion of the foam layers at a location in a region of the center of the sternum, the sound generating unit being configured to generate an acoustic signal when the portion of the foam layers is compressed with a force that sufficient for CPR.

Furthermore, a CPR training manikin is known from US 2007/0264621 A1, in which CPR is simulated by applying pressure to the body of the training manikin via a pneumatic or hydraulic piston.

Furthermore, US 2017/0193857 A1 describes how the elasticity of the body of a CPR training manikin can be controlled by rubber bands and a cardiopulmonary simulation can be carried out. However, the publication does not contain any information regarding the problem of the compression depth and the required damping properties of the material of the training manikin.

In addition, US 2018/0322808 A1 discloses a training manikin for simulating cardiopulmonary resuscitation, having a very robust structure which comprises a foam body which is provided with a cover or imprint in the form of a human torso with or without a head and which has a signal-generating unit within the foam body, the foam body consisting of a singular foam, the signal-generating unit being arranged in a region underneath the cover or imprint which corresponds to the sternum of the torso with or without a head, and the signal-generating unit emitting a signal when the sternum region of the imprint is compressed to a compression depth of 4 to 5 cm. The publication does not mention the emission of a signal only in the case of a corresponding compression duration of 100 ms to 1000 ms.

A sensor system for monitoring resuscitation exercises that can be inserted into a training manikin is known from U.S. Pat. No. 5,540,592 A. In this case, both the compression depth and the duration of the compressions can be recorded and evaluated. The emission of an optical signal by means of LEDs or lasers is not described.

EP 3 370 220 A1 describes a system for testing cardiopulmonary functions, a signal-generating unit showing the result of the tests regarding a sufficient compression depth and frequency on a display. However, no manikin is disclosed as a training device in this document, and an optical signal-generating unit in the form of LEDs or lasers is also not described.

The problem with the training manikins and the sensor systems from the known prior art is that the materials and the design and the use thereof in a CPR manikin are expensive, and their design and their proper use are time-consuming.

The problem addressed by the invention is therefore to develop an inexpensive CPR training manikin which can be used in large numbers (>10,000), for example in schools and other institutions, in an inexpensive and unproblematic manner.

In order to solve the problem, the training manikin according to the invention should have as few different materials as possible and in particular consist of only one foam compound, the foam necessarily having such damping properties and elastic properties that the compression depth required for CPR is reached at a certain application of pressure.

The problem is solved in the present case by the features of independent claim 1.

In the event of a circulatory arrest, it is imperative to act quickly: after approximately just three to five minutes, irreversible damage occurs in the brain due to the lack of oxygen supply. With cardiac massage, the residual oxygen in the blood can circulate to the brain, such that the probability of survival can be significantly increased until the emergency service arrives. Even without first aid knowledge, it is possible for the medical layperson to save lives. For this purpose, the sternum has to be repeatedly pressed firmly in the center of the chest.

The subject matter of the invention therefore comprises, as a training device for CPR, a foam body as a base, which foam body is provided with a schematic structure in the form of a human torso with or without a head. The foam body in this case consists of only a singular foam, i.e. the foam body is composed of only one foam compound or one foam layer. Such an embodiment of the subject matter of the invention is provided in order to minimize the costs for the training device and to simplify the handling of the training device as much as possible; in particular, no damage to portions of the training device should result in the device becoming completely unusable if damaged. The singular foam compound can, however, also have regions having different compression hardnesses, i.e. it is possible for the foam compound to be stronger or weaker laterally or proximally on the foam body. In particular, the compression hardness in the region of the compression point for a cardiopulmonary resuscitation measure can vary with respect to the rest of the foam body. It is thus possible for the foam body to consist of a foam compound which, however, is softer at the edges and harder in the center, in particular in the region of a marking for a cardiac massage. The foam body can accordingly also consist of two elements having different compression hardnesses, wherein the foam compounds are the same.

Since CPR training in particular requires that a compression having a certain compression strength or compression depth is exerted on the cardiac region in the center of the rib cage (in the region of the sternum), according to the invention, a signal-generating unit is arranged within the foam body, which unit is located underneath the imprint in the region of the sternum of the torso with or without a head and, from a certain compression depth corresponding to a certain compression strength, emits an acoustic and/or optical signal and/or indicates that a pressure pulse sufficient for CPR was exerted on the center of the chest. In this case, the compression depth or compression strength has to be maintained over a certain time interval. The signal from the signal-generating unit should be transmitted from a compression depth of 3 cm to 8 cm, with a compression duration in a time interval of 100 ms to 1000 ms. A signal for a deeper compression depth and longer compression duration can in this case be used to simulate CPR on stronger people, while a signal for a lower compression depth and shorter compression duration can be used to simulate CPR on the body of a child or a very slim person, in order to take the respective physiological or anatomical conditions into account.

In an advantageous embodiment of the subject matter of the invention, the signal-generating unit emits a signal at a compression depth of 5 cm to 6 cm and the compression duration should comprise a time interval of 400 ms to 600 ms in order to simulate a pressure pulse which is sufficient for CPR on the center of the chest. Such an embodiment of the subject matter of the invention can be used in particular for standardized simulation of a CPR on the body of an average adult.

In a special embodiment, the foam body, as a base, should be cushion-shaped or rectangular and, for example, have dimensions of the foam body in a range of 30 cm×50 cm×7 cm, it being possible for the edges of the cushion-shaped or rectangular foam body to be radially rounded. The design of the base of the subject matter of the invention as a small cushion or small rectangle is based on easy handling of a cushion-shaped or rectangular structure, be it in order to be able to easily hold the training manikin in the hands when in use or to store it in an easily stackable manner at a storage location when not in use. The shape zo and size are also suitable for transport or shipping which is as inexpensive as possible. To prevent injuries to the user of the training manikin according to the invention, the edges of the foam body can be rounded. For this purpose, the edges could be flanged or folded inward laterally from the outside, as a result of which a radially circumferential chamfer is generated. However, it is also possible to design the lateral regions of the foam body to be edge-shaped or to leave said regions edge-shaped in order to ensure that the CPR manikin is produced as inexpensively as possible.

The foam body of the training manikin according to the invention should have a compression hardness in a gross weight of 30 to 50, which should ensure that the compression depth in a range of 3 cm to 8 cm can be reached such that a compression depth of approximately ⅓ of the chest diameter is reached with a force of 3.0 kPa to 5.0 kPa. The gross weight indicates how many kilograms of material was used per cubic meter of foam. This amount determines the dimensional stability and durability of the foam. The compression hardness (kPa) indicates how much force is necessary to compress the foam by approx. 40% (of the normal height).

In order to ensure an elastic compression depth, the foam body according to the invention consists of a thermoplastic, elastomeric or duroplastic foam. The types of foam include, for example, but are not limited to, polystyrene, polypropylene and polyvinyl chloride, polyurethane, acrylonitrile butadiene and phenoplasts.

According to the invention, the foam body also has a cover or imprint which comprises the torso, with or without a head, of a human body. The cover can be designed as a cushion cover which completely encloses the foam body and to which an imprint of a human torso, with or without a head, is applied, or the schematic structure of a torso with or without a head is embedded or woven into the structure of the cushion cover. The cover can be provided with a zip fastener, a hook and loop fastener or buttons, which are arranged on the cover and ensure that it is fixed to the foam body. An imprint can also be arranged directly on the surface of the foam body, the schematic structure of a body being embossed in the sense of a blind embossing, or regions of different colors which represent a torso with or without a head being embedded in the foam body. The entire imprint or parts of the imprint, however, can also be applied to the surface of the foam body in the form of a film; wherein the film that can be applied and can consist in particular of thermoplastic elastomers based on urethane (TPU). In the present case, application is to be understood to mean a lamination-like application of an imprint or parts of an imprint, it being possible for the material to be applied to be a film which has regions having elements of a torso with or without a head, which forms the surface of the foam body.

Since, for training purposes for efficient CPR, the training manikin has to undergo a certain compression depth which is as localized as possible in the region of the imprinted or applied sternum, the manikin has a marking in the region of the sternum that can, for example, have a square, circular or heart-shaped cross section which is intended to indicate the contact surface for the hands during a cardiac massage. The size of the marking is variable and depends, if necessary, on the particular embodiments, in particular the size and compression hardness of the foam body of the training manikin, as well as on the physical properties of the users, in particular their size and age.

The training manikin has an analog or digital signal-generating unit underneath the schematic structure in the region of the sternum, which signal-generating unit emits or displays a corresponding signal when a corresponding compression depth is reached. According to the invention, an analog or digital signal-generating unit is to be understood to be a bell or a sensor with audio functions or a sensor which transmits a signal to a technical evaluation unit, for example by means of an app.

It is also possible to design the training manikin in the region underneath the schematic structure in the region of the sternum to have an analog or digital signal-generating unit, with a light bulb, an LED light or a laser light lighting up as a corresponding signal when a corresponding compression depth is reached. The light-emitting lamps are in this case embedded in the foam body and can also be present in a training manikin in combination with the devices for generating analog or digital sound signals.

Due to the design of the training manikin according to the invention in the sum of its features, it is particularly suitable as an inexpensive and easily manageable training device for cardiopulmonary resuscitation exercises.

The invention is explained again on the basis of the following embodiment:

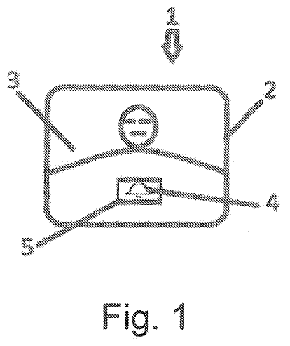

FIG. 1 shows a schematic plan view of a training manikin 1 for simulating cardiopulmonary resuscitation, in which a rectangular or cushion-shaped foam body 2 having a cover 3 or imprint 3 in the form of a human torso with or without a head can be seen as the base. In the central region of the cover 3 or imprint 3, a region is indicated with a marking 5, which region is intended to be the region of the sternum that is intended to be used as a contact surface for the hand or hands when a cardiac massage is performed. A signal-generating unit 4 is shown schematically within the foam body 2, which signal-generating unit emits a sound signal and/or light signal when a corresponding compression depth in a range of 3 cm to 8 cm is reached in the sternum region of the cover 3 or imprint 3 with a compression duration of 100 ms to 1000 ms.

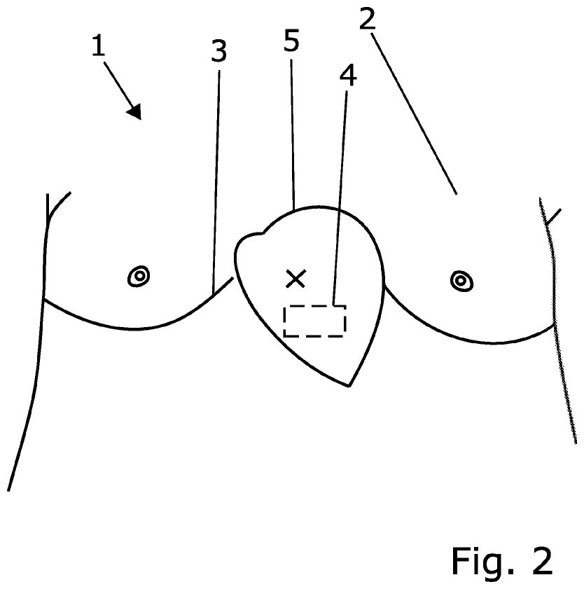

FIG. 2 shows a sketch of the region of the torso, without a head, of the training manikin 1, the marking 5 being designed as a heart-shaped contact surface for the hand or hands for a cardiac massage. In the present case, the image of the torso is designed as an imprint 3. The foam body 2 of the training manikin 1 is designed as a singular foam compound, but can also have regions having different compression hardnesses, i.e. it is possible for the foam compound to be stronger or weaker laterally or proximally on the foam body 2. In particular, the compression hardness in the region of the marking 5 for a cardiac massage can vary with respect to the rest of the foam body. A signal-generating unit 4 is arranged underneath the marking 5 of the foam body 2, which signal-generating unit emits a sound signal and/or light signal when a corresponding compression depth in a range of 3 cm to 8 cm is reached in the sternum region with a compression duration of 100 ms to 1000 ms. In the present case, the ideal compression point for the cardiac massage is marked with an x within the marking 5.

### LIST OF REFERENCE SIGNS

1 Training manikin
2 Foam body
3 Imprint /cover
4 Signal-generating unit
5 Mark

1. A training manikin for simulating cardiopulmonary resuscitation, the training manikin comprising:
a foam body which supports a schematic structure of a human torso with or without a head and consists of a singular foam, a signal-generating unit being arranged inside the foam body, which the signal-generating unit is located in the foam body in a region underneath the sternum of the torso with or without a head and emits a signal when the sternum region of the torso with or without a head is compressed to a compression depth of 3 cm to 8 cm with a compression duration in a time interval of 100 ms to 1000 ms,
characterized in that
the schematic structure of the human torso with or without a head is designed as a cushion cover which completely encloses the foam body, the schematic structure of the torso with or without a head being embedded or woven into the structure of the cover and the cover being provided with a zip fastener or a hook and loop fastener or buttons, which are arranged on the cover and fix the cover to the foam body, or the schematic structure of the human torso with or without a head being applied to the foam body as an imprint or to the surface of the foam body as part of a film, or the schematic structure of the human torso with or without a head being embossed as a blind embossing in the foam body.

2. The training manikin according to claim 1, characterized in that the signal-generating unit emits a signal in the case of a compression depth of 5 cm to 6 cm in the sternum region of the image of the human torso, with or without a head, of the foam body with a compression duration in a time interval of 400 ms to 600 ms.

3. The training manikin according to claim 1, characterized in that the foam body is rectangular and the dimensions of the body are in a range of between 30 cm×50 cm×7 cm, the edges of the rectangular foam body being radially rounded.

4. The training manikin according to claim 1, characterized in that the foam body has a compression hardness in a gross weight of 30 to 50, regions of the foam body varying laterally in compression hardness with respect to proximal regions in compression hardness.

5. The training manikin according to claim 1, characterized in that the foam body comprises thermoplastic foams or elastomeric foams or duroplastic foams, regions of different coloration being present.

6. The training manikin according to claim 5, characterized in that the foams of the foam body are selected from a group of compounds comprising polystyrene, polypropylene and polyvinyl chloride, polyurethane, acrylonitrile butadiene and phenol formaldehyde resins.

7. The training manikin according to claim 1, characterized in that the torso with or without a head is laminated onto the surface of the foam body as part of a film, the film consisting of thermoplastic elastomers based on urethane (TPU).

8. The training manikin according to claim 1, characterized in that the region of the torso with or without a head that corresponds to the sternum has a marking.

9. The training manikin according to claim 8, characterized in that the marking has a square, circular or heart-shaped cross section as a hand rest surface for cardiac massage.

10. The training manikin according to claim 1, characterized in that the signal-generating unit generates analog or digital sound signals.

11. The training manikin according to claim 10, characterized in that the signal-generating unit comprises a bell or a sensor having audio functions.

12. The training manikin according to claim 1, characterized in that the signal-generating unit generates optical signals.

13. The training manikin according to claim 12, characterized in that the signal-generating unit is a light bulb, an LED light or a laser.

14. The training manikin according to claim 1, characterized in that the signal-generating unit transmits a signal to a technical evaluation unit in the form of an app.

15. A method, the method comprising:
performing cardiopulmonary resuscitation exercises on a training manikin, wherein the training manikin comprises:
a foam body which supports a schematic structure of a human torso with or without a head and consists of a singular foam, a signal-generating unit being arranged inside the foam body, which the signal-generating unit is located in the foam body in a region underneath the sternum of the torso with or without a head and emits a signal when the sternum region of the torso with or without a head is compressed to a compression depth of 3 cm to 8 cm with a compression duration in a time interval of 100 ms to 1000 ms,
characterized in that
the schematic structure of the human torso with or without a head is designed as a cushion cover which completely encloses the foam body, the schematic structure of the torso with or without a head being embedded or woven into the structure of the cover and the cover being provided with a zip fastener or a hook and loop fastener or buttons, which are arranged on the cover and fix the cover to the foam body, or the schematic structure of the human torso with or without a head being applied to the foam body as an imprint or to the surface of the foam body as part of a film, or the schematic structure of the human torso with or without a head being embossed as a blind embossing in the foam body.
